(19) United States
(12) Patent Application Publication
(10) Pub. No.: US 2016/0213268 A1
(43) Pub. Date: Jul. 28, 2016

(54) DEVICE FOR DETECTING A SPIKE IN ONE OF A PLURALITY OF NEURAL SIGNALS

(71) Applicant: AGENCY FOR SCIENCE, TECHNOLOGY AND RESEARCH, Singapore (SG)

(72) Inventors: Seong-Jin KIM, Singapore (SG); Minkyu JE, Singapore (SG)

(21) Appl. No.: 14/917,932

(22) PCT Filed: Sep. 9, 2014
(86) PCT No.: PCT/SG2014/000425

§ 371 Date: Mar. 9, 2016

(30) Foreign Application Priority Data
Sep. 9, 2013 (SG) 201306771-5

(51) Int. Cl. A61B 5/04; G01R 23/00

(52) U.S. Cl. CPC A61B 5/04001; G01R 23/00

(57) ABSTRACT

A method for determining an occurrence of a spike in one of a plurality of neural signals is provided, the spike relating to an action potential in the one of the plurality of neural signals. The method includes sampling the plurality of neural signals at a sampling frequency; extracting frequency features from each of the plurality of neural signals during sampling of the plurality of neural signals; and monitoring the extracted frequency features to determine characteristics of the one of the plurality of neural signals indicative of an occurrence of a spike.

### PRIORITY CLAIM

The present application claims priority to Singaporean Patent Application No. 201306771-5, filed on 9 Sep. 2013.

### FIELD OF INVENTION

The present invention relates broadly to neural signal monitoring and more particularly to a device for detecting a spike in one of a plurality of neural signals.

### BACKGROUND

An important aspect of research related to neuroscience is the detection and analysis of neural signals. Neural signals relate to electrically excitable cells that process and transmit information through electrical and chemical signals. With current technology, recordings have been made of electrical neurons in neural networks such as the brain, including the human brain, cell cultures and in vivo tissue.

In order to record neural signals, a neural signal recording device has to be utilized. Conventionally, in order to acquire signals from different neurons in a region of the brain, a recording device could be implanted in the brain for recording neural signals. Another conventional technique to acquire signals is to position one or more electrode sensors on a subject's brain for recording neural signals.

It is of particular importance to detect an occurrence of a spike during recording of the neural signals. A spike represents an action potential that typically results from a spontaneous or stimulus-evoked activity. As such, a spike can be seen as a sharp transient that is visibly different from background noise. Since the spike is a rare event in neural signals, it is desirable to detect the spike in order to minimize power dissipation while preserving the essential neural activity.

However, conventional spike detection methods and devices typically suffer from shortcomings that prevent their use in low-power devices of sufficiently small size for implanting in a subject. A typical conventional spike detection technique makes use of amplitude detection in the neural signals and a shortcoming of such technique is that it does not perform adequately since it has been proven to lack robustness to DC shifts.

FIG. 1 shows a typical neural signal according to R. Muller, S. Cambini, and J. M. Rabaey, “A 0.013 mm², 5 μW, DC-Coupled Neural Signal Acquisition IC with 0.5V Supply,” IEEE J. Solid-State Circuit, published in 2012. The neural signal is categorized to two different potentials. One is a local field potential (LPF) which is an averaged electrophysiological signal caused by the electrical current flowing from all dendritic synaptic activity within a volume of tissue. The other is an Action Potential (AP), also known as the spike that is generated by each neuron. The LPF has relatively low frequency from 1 to 300 Hz, while the spike is represented in 300 to 10 KHz. In order to eliminate DC offset and separate the spike from the LPF, conventional neural recording system makes use of high-pass or band-pass filter as shown in FIG. 2(a).

FIG. 2(a) shows a conventional neural recording system. The system includes a low-noise amplifier (LNA) for amplifying sampled signals from a sample and holder device (S/H), an analog-to-digital converter (ADC) and a radio integrated circuit to transmit digitized signal.

FIGS. 2(b) and 2(c) show a conventional neural recording system including a spike detector in analog and digital domains, respectively. The spike detectors shown in FIGS. 2(b) and 2(c) compare the signal potential to threshold potential. However, the absolute values of the spike potentials may vary depending on the sites on where the electrodes are positioned. For example, electrodes positioned far away from a neuron in a subject's brain may detect a different spike potential than the electrodes positioned close to a neuron in the subject's brain. As such, this technique may deteriorate the accuracy of the spike detection in a plurality of neural signals.

Thus, what is needed is a method and device for detecting a spike in one of a plurality of neural signals by using other characteristics together with the amplitude information, which minimizes power dissipation and increases its accuracy. Furthermore, other desirable features and characteristics will become apparent from the subsequent detailed description and the appended claims, taken in conjunction with the accompanying drawings and this background of the disclosure.

### SUMMARY

According to the Detailed Description, a method for determining an occurrence of a spike in one of a plurality of neural signals is provided, the spike relating to an action potential in the one of the plurality of neural signals. The method includes sampling the plurality of neural signals at a sampling frequency; extracting frequency features from each of the plurality of neural signals during sampling of the plurality of neural signals; and monitoring the extracted frequency features to determine characteristics of the one of the plurality of neural signals indicative of an occurrence of a spike.

In accordance with another aspect, a device for determining an occurrence of a spike in one of a plurality of neural signals is provided, the spike relating to an action potential in the one of the plurality of neural signals. The device includes a sampling unit and a spike detector. The sampling unit is adapted to sample the plurality of neural signals at a sampling frequency. The spike detector is coupleable to the sampling unit and adapted to extract frequency features from each of the plurality of neural signals during sampling of the plurality of neural signals. The spike detector is further adapted to monitor the extracted frequency features to determine characteristics of the one of the plurality of neural signals indicative of an occurrence of a spike.

### BRIEF DESCRIPTION OF THE DRAWINGS

The accompanying figures where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 shows a typical neural signal.

FIG. 2(a) shows a conventional neural recording system.

FIG. 2(b) shows a conventional neural recording system including a spike detector in an analog domain.

FIG. 2(c) shows a conventional neural recording system including a spike detector in a digital domain.

FIG. 3 shows a block diagram of a device for detecting a spike in one of a plurality of neural signals in accordance with an embodiment.

FIG. 4 shows a schematic diagram of the delta sampling architecture used in FIG. 3.

FIG. 5 shows simulation timing waveforms of the delta sampling architecture shown in FIG. 4.

FIG. 6 shows some sampling points on a representative neural signal.

FIGS. 7(a)-7(c) show schematic diagrams of the device shown in FIG. 1 in accordance with example embodiments.

FIG. 8(a) shows a schematic diagram for detecting an amount of change in at least two consecutive signals based on charge redistribution when there is a positive sign in accordance with an example embodiment.

FIG. 8(b) shows a schematic diagram for detecting an amount of change in at least two consecutive signals based on charge redistribution when there is a negative sign in accordance with an example embodiment.

FIG. 9 shows simulation results of the delta sampling architecture shown in FIG. 4.

FIG. 10 shows simulation results of the device shown in FIG. 3 with a spike.

FIG. 11 shows simulation results of the device shown in FIG. 3 without a spike.

### DETAILED DESCRIPTION

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

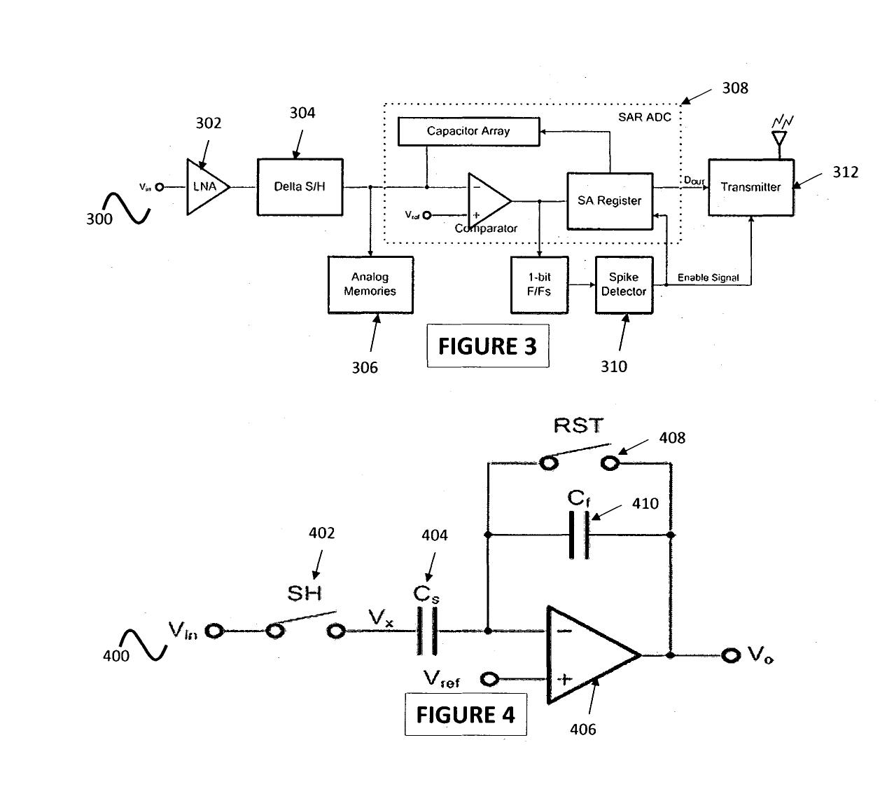

FIG. 3 shows a block diagram of a device 300 for detecting a spike in one of a plurality of neural signals in accordance with an embodiment. The device 300 includes a low-noise amplifier (LNA) 302 coupled to a delta sample and holder (delta S/H) or delta sampling architecture 304 in an example embodiment. The LNA 302 is adapted to amplify analog signals for inputting into the delta S/H 304.

A typical S/H, also “track-and-hold” device, is an analog device that samples (captures, grabs) the voltage of a continuously varying analog signal and holds (locks, freezes) its value at a constant level for a specified minimum period of time. They are typically used together with ADC to eliminate variations in input signal that can corrupt the conversion process. In an embodiment, the delta S/H 304 is adapted to sample and hold the analog signals and extract amplitude and frequency information of the analog signals. Further, the delta S/H 304 is also adapted to achieve high-pass filtering functionality which eliminates the need to use a high-pass or band-pass filter typically used in a conventional neural recording system.

In an embodiment, the delta S/H 304 is coupled to analog memories 306. Analog memories store consecutive output signal of the delta S/H and the spike detector 310 recognizes a spike signal by analyzing stored data. Once the spike signal is detected, one of analog memories 306 is fed to an ADC 308. In the instance that the spike detector 310 extracts a spike in the analog signals, the spike detector 310 may be so controlled to trigger an enable signal to the ADC 308. In this manner, operations of the ADC 308 are controlled to be activated when a spike is detected.

The device 300 is configured to detect the spike during sampling of the analog signals. Advantageously, this allows the ADC 308 and a transmitter 312 to only consume electronic power necessary for the spike signal. This effectively results in minimizing power dissipation by reducing a dynamic range of neural signals.

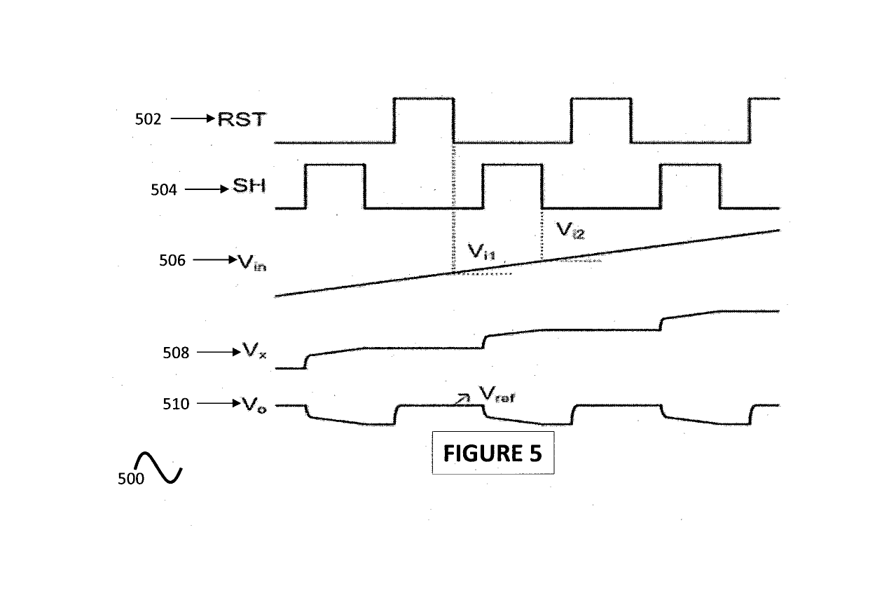

FIG. 4 shows a schematic diagram of the delta sampling architecture 400 used in FIG. 3. In an embodiment, the delta sampling architecture 400 includes a switch 402 coupled to a capacitor 404. The switch 402 is typically a field effect transistor switch and the capacitor 404 is used to store electric charges.

The capacitor 404 is coupled to at least one operational amplifier 406. The amplifier 406 is adapted to charge or discharge the capacitor 410 so that the voltage across the capacitor is practically equal or proportional to an input voltage.

In hold mode, the switch 402 disconnects the capacitor 404 from the amplifier 406 and the voltages across the capacitors 404 and 410 are maintained during the operation.

The delta sampling architecture 400 uses multiple feedback capacitors 404 and 410 that can function as analog memories. Advantageously, it helps to preserve a spike signal. On the other hand, conventional techniques typically lose data pertaining to neural signals before detecting a spike because feedback capacitors are not used as analog memories.

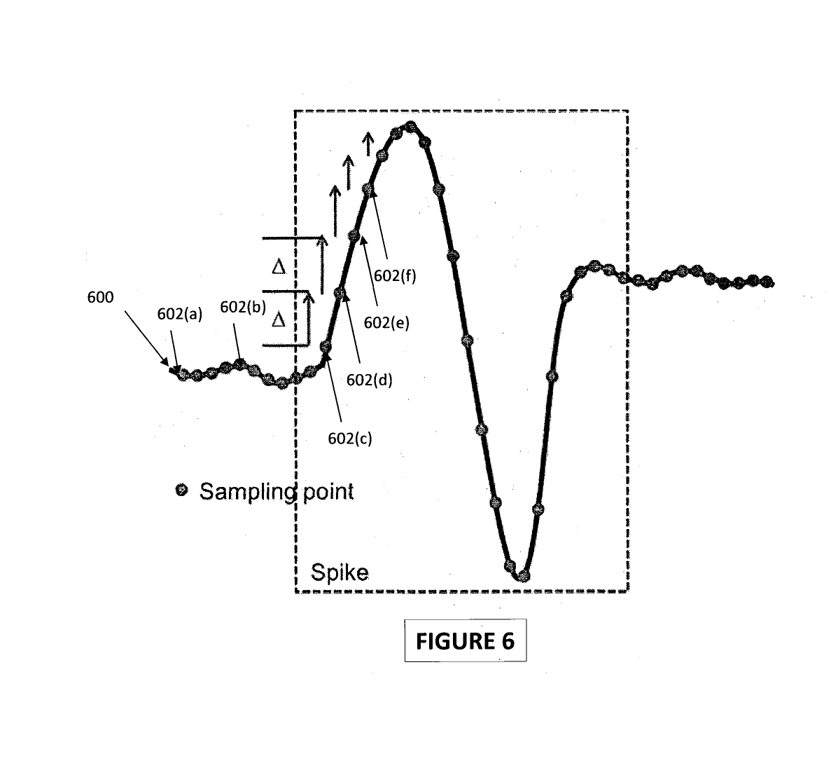

FIG. 5 shows simulation timing waveforms of the delta sampling architecture shown in FIG. 4. Waveform 502 shows a signal for RST switch 408 shown in FIG. 4. When the RST switch is turned on, the output of the delta S/H is reset to V_{REF}. After resetting the delta S/H, the output is changed by the sampled voltage Vₓ. Waveform 504 shows a signal for the S/H switch 402 shown in FIG. 4. Waveform 506 represents the input voltage Vᵢₙ. Waveform 508 represents the sampled voltage Vₓ in response to the S/H switch 402 operation. Waveform 510 represents the output voltage Vₒᵤₜ of the delta sampling architecture.

The output voltage Vₒᵤₜ of the delta sampling architecture is given by:

$$Δ {V}_{o} = {V}_{ref} - {V}_{o} = - \frac{{C}_{s}}{{C}_{f}} ( {V}_{i 1} - {V}_{i 2} ) = - \frac{{C}_{s}}{{C}_{f}} Δ {V}_{in} \qquad ( 1 )$$

Assuming that the input signal is sinusoidal wave and the gain is 1, the output voltage Vₒᵤₜ of the delta sampling architecture is given by:

$$\begin{matrix}Δ {V}_{o} = A sin ( 2 π {ft}_{k + 1} ) - A sin ( 2 {π ft}_{k} ) = 2 A cos ( 2 π f {t}_{0} ) sin ( π f Δ t ) & ( 2 ) \\ {t}_{k + 1} = {t}_{0} + Δ t \text{/} 2 & ( 3 ) \\ {t}_{k} = {t}_{0} - Δ t \text{/} 2 & ( 4 ) \\ max ( Δ {V}_{o} ) = 2 A sin ( π f Δ t ) = 2 A sin ( π \frac{f}{{f}_{s}} ) = 2 A sin ( \frac{π}{n} ) & ( 5 )\end{matrix}$$

where A and f are amplitude and frequency of the signal, and fₛ and n are sampling frequency and rate, respectively. The above equations show how delta sampling by the delta sampling architecture 400 can ignore low frequency component of the neural signals because its output level in high sampling rate should be lower than input referred noise. In an embodiment, the sampling frequency is chosen from 10 to 40 kHz. Advantageously, this prevents the spike signal from aliasing error. As such, the different signals will not become indistinguishable (or aliasing of one another). This also helps to preserve the spike detection.

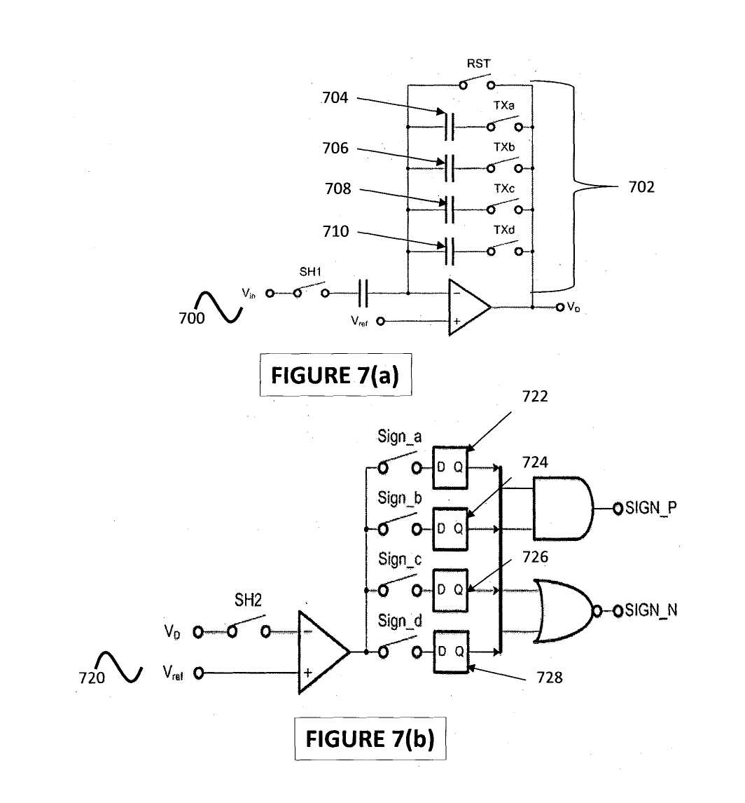

FIG. 6 shows some sampling points 602 on a representative neural signal 600. FIG. 6 shows how high frequency component can be suppressed by observing how the signal changes from successive recordings (or signals) from the delta sampling architecture. The factors that affect the signal change of the successive recordings include the sampling frequency and the number of samples. The observation of the successive recordings from the delta sampling architecture is indicative of the characteristics of the spike. As such, the signs of the consecutive recordings can be observed from the delta sampling architecture and utilized as a low pass filter. In order to increase the accuracy of the spike detection, it is necessary to discriminate the spike from noise.

FIG. 6 shows that the change from a sampling point 602(a) to a next consecutive sampling point 602(b) has a smaller amplitude (or amount of change) in comparison to the change from a sampling point 602(c) to sampling point 602(d). Points 602(c) and 602(d) are arranged on a rising portion of the signal 600 which represents a spike. As such, this shows that the amount of change between any two consecutive points in the spike is much larger than other points of the signal. In an embodiment, a spike detector is used to compare the amount of change between two consecutive points with a pre-determined value, thereby distinguishing the spike from noise.

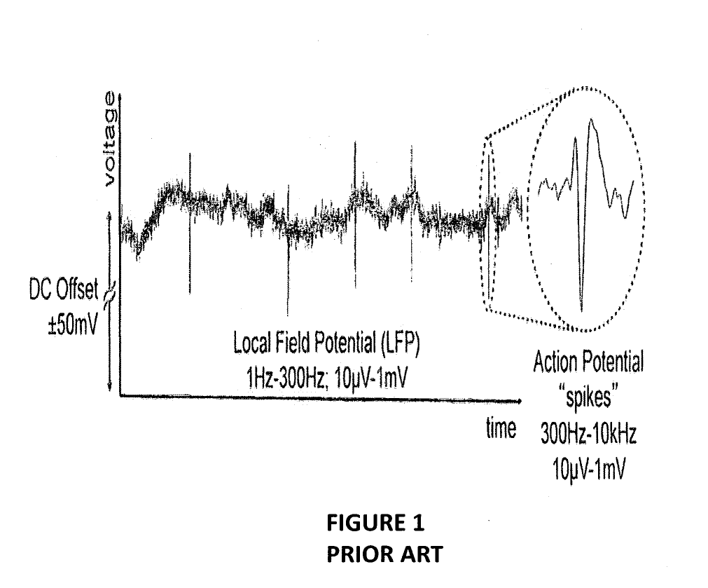

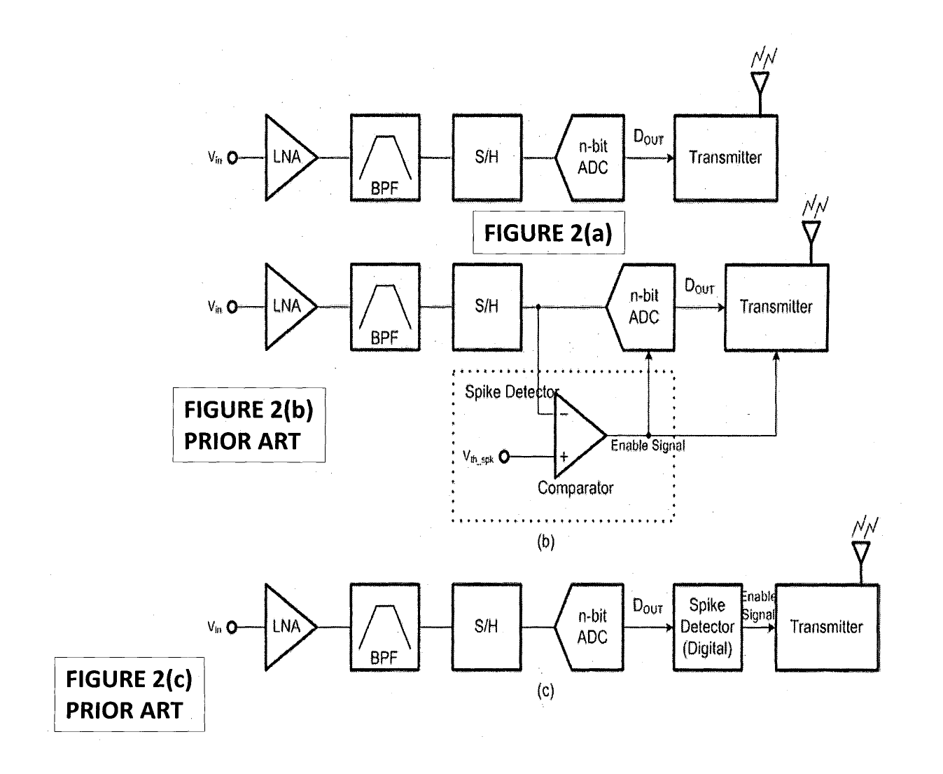

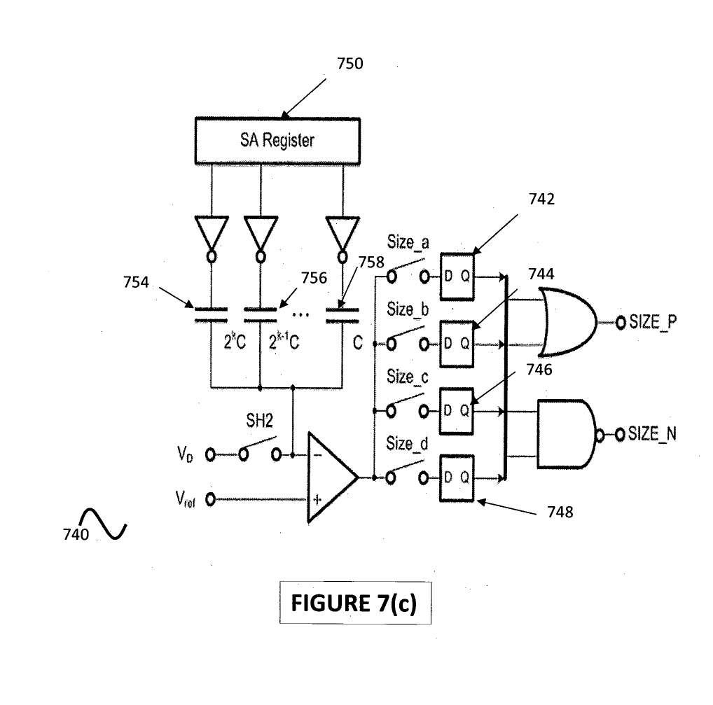

FIGS. 7(a)-7(c) show schematic diagrams of the device shown in FIG. 1 in accordance with example embodiments. FIG. 7(a) shows a device 700 including a delta sampling architecture 702. The delta sampling architecture 702 includes four feedback capacitors 704, 706, 708 and 710 as analog memories. Each of the plurality of feedback capacitors helps to detect a signal. Advantageously, having a plurality of feedback capacitors functioning as analog memories helps to preserve a spike signal. This cannot be achieved by the conventional techniques of detecting a spike because information pertaining to a spike is usually lost.

FIG. 7(b) shows a device 720 including four D flip-flops 722, 724, 726 and 728 for latching the sign of each data. The output of the sign detector SIGN_P or SIGN_N goes high when all of signs into the four D flip-flops 722, 724, 726 and 728 are either positive or negative. Advantageously, this makes it possible to detect a spike if four consecutive signals are having a same direction of change, e.g., all four signals are having a positive or negative direction of change.

FIG. 7(c) shows a device 740 including a size detector module 750. The size detector module 750 includes capacitors 754, 756 and 758 suitable for successive approximation ADC by charge redistribution. The successive approximation register or size detector module 750 is adapted to control the capacitors 754, 756 and 758 for generating pre-defined threshold level that is compared with each sampled data. Further, the device 740 includes four D flip-flops 742, 744, 746 and 748 for latching comparison result of each data, which represents the size of each data. Similar to the device shown in FIG. 7(b), the output of the size detector SIZE_P or SIZE_N goes high when the direction of change detected by the four D flip-flops 742, 744, 746 and 748 are either positive or negative.

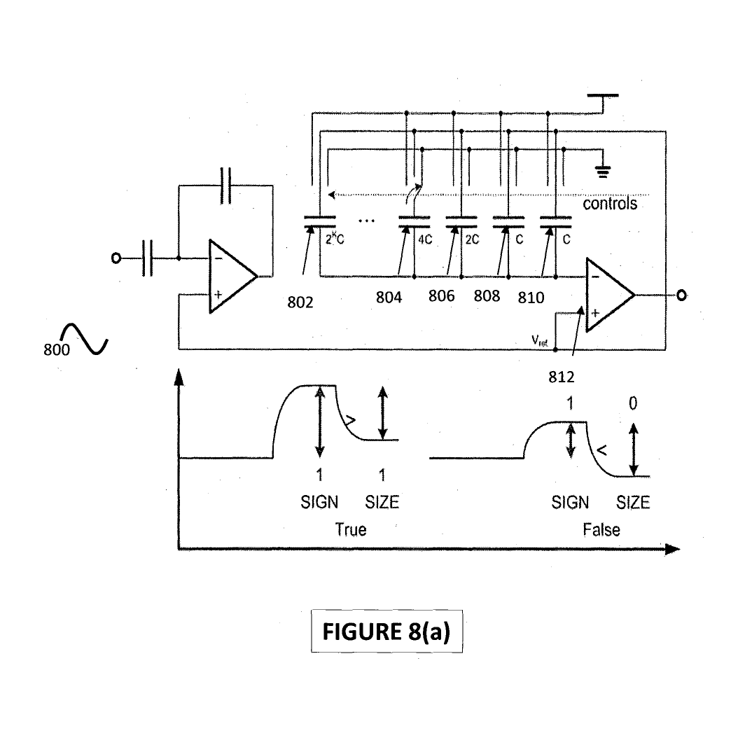

FIG. 8(a) shows a schematic diagram of a circuit 800 for detecting size by charge redistribution when there is a positive sign in accordance with an example embodiment. The circuit 800 includes a plurality of switches 802, 804, 806, 808 and 810. In the event that the result of the size detection is positive, one of the switches 802, 804, 806, 808 and 810 is changed from reference level to ground, thereby pushing down the potential in a negative node of the comparator 812.

FIG. 8(b) shows a schematic diagram of a circuit 850 for detecting size by charge redistribution when there is a negative sign in accordance with an example embodiment. The circuit 850 includes a plurality of switches 852, 854, 856, 858 and 860. In the event that the result of the size detection is negative, the charge redistribution increases the potential. As shown, the circuits 800, 850 shown in FIGS. 8(a) and 8(b) can detect the spike signal with both positive and negative peak by comparing the changed sample data (subtracted value between sampled data and charge-redistributed value) with the reference voltage.

A spike is considered detected if the change in several numbers of consecutive signals has the same sign and one of the signal changes is determined to be larger than the generated (or predetermined) potential level. In an embodiment, the number of consecutive signals is four. It is apparent to a person skilled in the art that any numbers of size and sign considerations are possible to determine a spike in a plurality of neural signals.

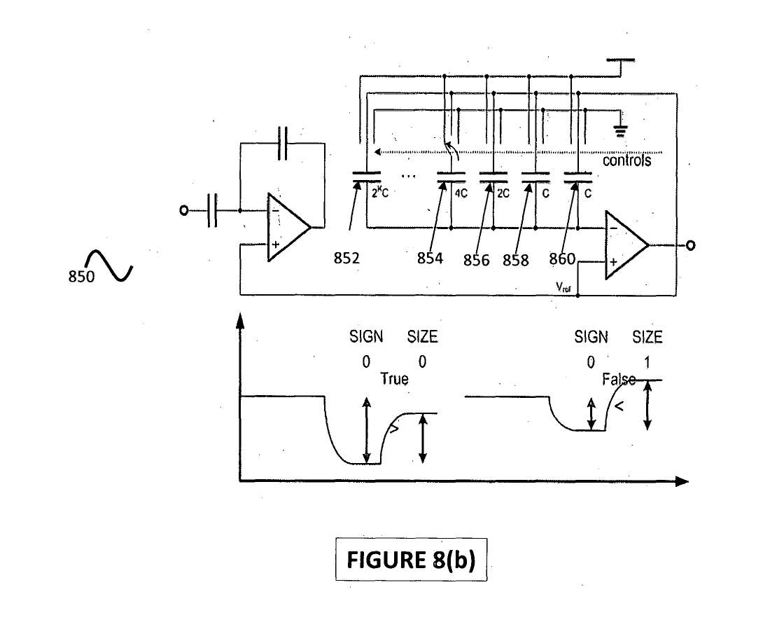

FIG. 9 shows simulation results 900 of the delta sampling architecture shown in FIG. 4. Simulation result 902 represents the input voltage V in a sine waveform. Simulation result 904 represents the output voltage Vₒᵤₜₚᵤₜ in a cosine waveform because the delta sampling architecture gives the 1^{st} order derivative of input signal which is also shown in equation 2.

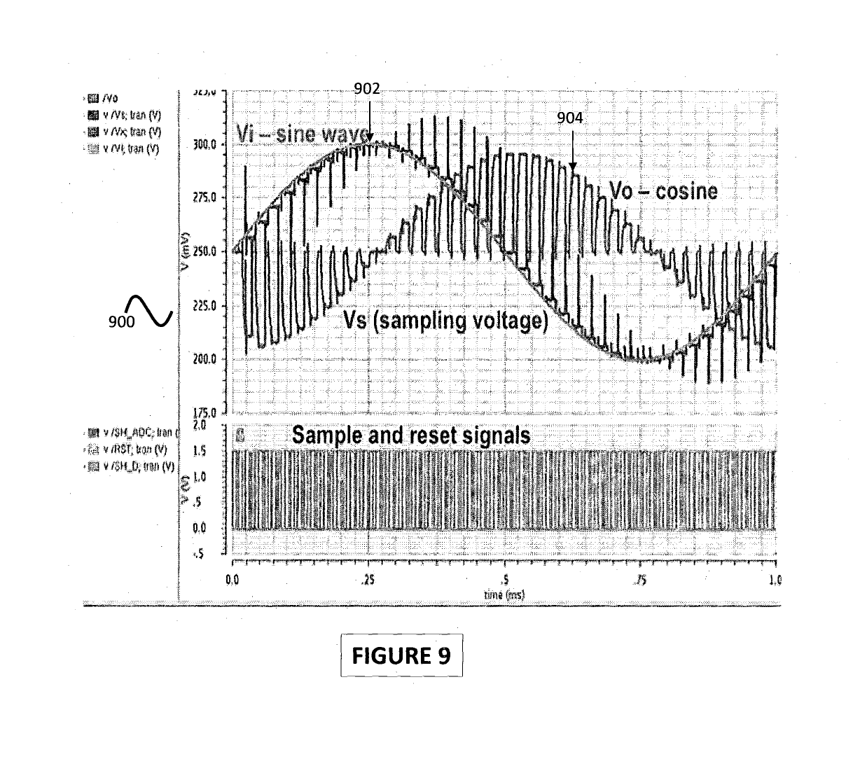

FIG. 10 shows simulation results 1000 of the device shown in FIG. 3 with a spike. An input spike 1002 represents a signal that is set to a sine waveform with 2 kHz frequency and 40 mV amplitude and it is fed into the device shown in FIG. 3. A gain of 100 is set to LNA 302 (FIG. 3) so that the spike has 400 μV amplitude. The sampling frequency is 42 kHz and the power output is set at 0.5V in this simulation.

A delta signal 1006 is represented by a cosine waveform and a delayed signal 1008 is shown. Three enable signals 1010, 1012 and 1014 are detected for triggering the operations in the ADC 308 shown in FIG. 3. As shown in FIG. 10, before the triggering of the first enable signal 1010, four consecutive output of the delta S/H have identical signs and the amount of signal change is large enough. As such, the detection result goes high and the ADC operation is triggered.

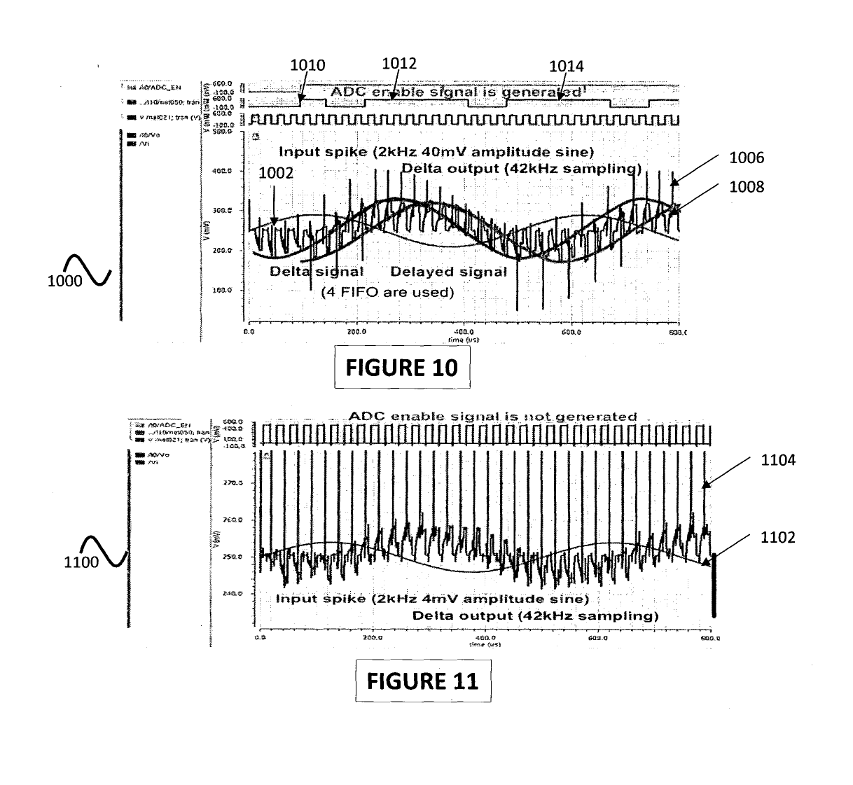

FIG. 11 shows simulation results 1100 of the device shown in FIG. 3 without a spike. An input spike 1102 and a delta signal 1104 are shown. As it can be seen, when the delta signal is not large enough to be considered as a spike (e.g., the amount of change in consecutive signals is not large enough), an enable signal will not be generated.

In order to generate the simulation results from the delta sampling architecture shown in FIG. 3, the power consumed is expected to be less than 0.2 nW/ch, without taking into consideration the LNA and the bias circuitry.

Accordingly, the above description presents a technique for detecting a spike that can achieve low power dissipation. This provides a spike detector having a low-power usage according to the proposed technique, making it safe to be used on a subject's head or to be implemented in the subject's brain, without sacrificing the essential recording of the neural signals.

The proposed technique measures consecutive signal changes and extracts frequency features from the plurality of neural signals. This helps to resolve the shortcoming of the conventional techniques which make use of only extracting amplitude features. Accordingly, the proposed technique solves the problem of lacking robustness to DC shifts posed by such amplitude extracting techniques.

In an embodiment, the proposed technique extracts both amplitude and frequency features from the plurality of neural signals. Beneficially, since the proposed technique extracts and monitors frequency and amplitude features from each of the plurality of neural signals, it is able to improve the accuracy in spike detection even when signals with different amplitude are received in a probe.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

1. A method for determining an occurrence of a spike in one of a plurality of neural signals, the spike relating to an action potential in the one of the plurality of neural signals, the method comprising:
sampling the plurality of neural signals at a sampling frequency;
extracting features from each of the plurality of neural signals during sampling of the plurality of neural signals, the features being frequency features;
monitoring the extracted frequency features to determine characteristics of the one of the plurality of neural signals indicative of the occurrence of the spike, and
sending the one of the plurality of neural signals to an analog-to-digital converter (ADC) when the occurrence of the spike is determined,
wherein the monitoring comprises checking a plurality of consecutive signals from the extracted frequency features and determining the occurrence of the spike when an amount of change between two consecutive neural signals of the plurality of consecutive neural signals is greater than a predetermined potential level or when the plurality of consecutive neural signals all have a same direction of signal change.

2. The method according to claim 1, wherein the operation of extracting features from each of the plurality of neural signals during sampling of the plurality of neural signals includes extracting the frequency features and amplitude features from the plurality of neural signals and monitoring the extracted amplitude features to determine characteristics of the one of the plurality of neural signals indicative of the occurrence of the spike.

3. The method according to claim 2, wherein the monitoring of the extracted amplitude features is performed on two consecutive neural signals.

4. The method according to claim 1, wherein the sampling frequency is from 10 kHz-40 kHz.

5. A device for determining an occurrence of a spike in one of a plurality of neural signals, the spike relating to an action potential in the one of the plurality of neural signals, the device comprising:
a sampling unit adapted to sample the plurality of neural signals at a sampling frequency;
a spike detector coupleable to the sampling unit and adapted to extract features from each of the plurality of neural signals during sampling of the plurality of neural signals, the features being frequency features, the spike detector further adapted to monitor the extracted frequency features to determine characteristics of the one of the plurality of neural signals indicative of an occurrence of a spike; and
an analog-to-digital converter (ADC), wherein the spike detector is configured to send the one of the plurality of neural signals to the ADC when the occurrence of the spike is determined,
wherein during monitoring the spike detector is configured to check a plurality of consecutive signals from the extracted frequency features and determine the occurrence of the spike when an amount of change between two consecutive neural signals of the plurality of consecutive neural signals is greater than a predetermined potential level or the plurality of consecutive neural signals all have a same direction of signal change.

6. The device according to claim 5, wherein the sampling unit is coupleable to one or more analog memories.

7. The device according to claim 5, wherein the spike detector is further adapted to
extract the frequency features from the plurality of neural signals;
extract amplitude features from the plurality of neural signals; and
monitor the extracted amplitude features to determine the occurrence of the spike, the extracted amplitude features indicative of characteristics of the one of the plurality of neural signals when there is an occurrence of a spike.

8. The device according to claim 5, wherein the spike detector is further adapted to monitor the extracted amplitude features on two consecutive signals.

9. The device according to claim 5, wherein the sampling frequency is from 10 kHz-40 kHz.

10. The device according to claim 5, wherein the spike detector comprises at least one feedback capacitor.
